(19) United States
(12) Patent Application Publication
(10) Pub. No.: US 2025/0022462 A1
(43) Pub. Date: Jan. 16, 2025

(54) VOICE COMMAND INTEGRATED KEYBOARD FOR MEDICAL RECORD ACCESS

(71) Applicants: Philip A. D'Agostino, Greenwood Village, CO (US); Jason A. Dell, Highlands Ranch, CO (US); Ashley A. Bye, St. Louis Park, MN (US); Pradeep Kumar, Bangalore (IN)

(72) Inventors: Philip A. D'Agostino, Greenwood Village, CO (US); Jason A. Dell, Highlands Ranch, CO (US); Ashley A. Bye, St. Louis Park, MN (US); Pradeep Kumar, Bangalore (IN)

(73) Assignee: DOC BUDDY INC., Greenwood Village, CO (US)

(21) Appl. No.: 18/771,020

(22) Filed: Jul. 12, 2024

Related U.S. Application Data
(60) Provisional application No. 63/526,216, filed on Jul. 12, 2023.

(51) Int. Cl. G10L 15/22; G06F 3/04886; G16H 10/60; G16H 15/00

(52) U.S. Cl. CPC G10L 15/22; G06F 3/04886; G16H 10/60; G16H 15/00

(57) ABSTRACT

A medical dictation application configured to operate on a computing device includes a dictation component and a customized operating system (OS) feature in communication with the dictation component, where the customized OS feature and the dictation component utilize a shared authentication process of the medical dictation application, where the customized OS feature is inaccessible to a user device prior to authentication of the user device with the medical dictation application utilizing the shared authentication process.

### CROSS-REFERENCE TO RELATED APPLICATIONS

This Application claims the benefit of priority to U.S. Provisional Application No. 63/526,216, filed Jul. 12, 2023, and titled “VOICE COMMAND INTEGRATED KEYBOARD FOR MEDICAL RECORD ACCESS,” the disclosure of which is incorporated by reference herein in its entirety.

### BACKGROUND

Various customized operating system (OS) features may be useful for performing various functions using mobile devices, and may be useful across different mobile applications. For example, keyboard extensions may be useful for medical dictation using mobile devices. For example, inclusion of medical terminology in such keyboards may improve dictation. However, existing keyboard extensions or other types of customized operating system features may pose security risks due to the need to transmit data between a medical dictation application and the keyboard. Such keyboard extensions may further be difficult to configure on an individual user basis and may time out during longer dictations.

### SUMMARY

A medical dictation application configured to operate on a computing device includes a dictation component and a customized operating system (OS) feature in communication with the dictation component, where the customized OS feature and the dictation component utilize a shared authentication process of the medical dictation application, where the customized OS feature is inaccessible to a user device prior to authentication of the user device with the medical dictation application utilizing the shared authentication process.

In some examples, the customized OS feature is a keyboard extension, where the keyboard extension provides at least one dictation shortcut for inserting text into a dictation responsive to a voice command provided using the keyboard extension.

In some examples, an interface to the keyboard extension at the user device is persistent, such that the keyboard extension executes on the user device until a command is provided to exit the keyboard extension or until a predetermined time out period has elapsed.

In some examples, the customized OS feature and the dictation component utilize shared data within the medical dictation application, where the shared data includes patient data.

In some examples, the customized OS feature and the dictation component utilize shared data within the medical dictation application, where the shared data includes instructions to access patient data from an external medical system.

In some examples, a second application separate form the medical dictation application is in communication with the customized OS feature.

A computer implemented method includes receiving, from a user device within a first software application, a request to access a keyboard extension of a medical dictation application, wherein the keyboard extension is accessible within a user interface of the first software application and in communication with a dictation component of the medical dictation application. The method further includes receiving, at an interface to the keyboard extension, a voice command, where the voice command includes a dictation shortcut for inserting text into a dictation within the first software application and inserting the text into the dictation within the first software application.

In some examples, the text is inserted into the dictation by copying text from the interface to the keyboard extension within the medical dictation application to the first software application using a cursor position determined with the request to access the keyboard extension of the medical dictation application.

In some examples, the interface to the keyboard extension is displayed within the medical dictation application responsive to authenticating the user device at the medical dictation application.

In some examples, the keyboard extension and the dictation component utilize shared data, where the method further includes accessing, by the keyboard extension responsive to the voice command, the shared data.

In some examples, they keyboard extension and the dictation component utilize shared data, where the shared data includes instructions to access patient data from an external medical system.

In some examples, the interface to the keyboard extension is persistent, such that the interface to the keyboard extension executes on the user device until a command is provided to exit the keyboard extension or until a predetermined time out period has elapsed.

In some examples, the keyboard extension extends a keyboard of the user device, where the voice command, when received, is not shared with the keyboard of the user device or additional extensions to the keyboard of the user device.

A computer implemented method for medical dictation includes authorizing a user device for access to a dictation application and launching the dictation application based on a command received from a user interface (UI) feature via a second application different from the dictation application, where the UI feature is accessible in both the dictation application and the second application. The method further includes receiving voice commands at the dictation application, translating the voice commands into text by the dictation component, and transmitting the text to the second application via the UI feature.

In some examples, the UI feature is further accessible in a third application different from the dictation application and the second application.

In some examples, the method further includes saving a cursor location of user activity within the second application and transmitting the text to the saved cursor location.

A method for medical dictation includes launching a medical dictation application by selecting a UI feature accessible within a second application, where the second application is different from the medical dictation application and receiving input by the medical dictation application via the UI feature. The method further includes translating the input into medical text by the medical dictation application, providing the medical text to the UI feature, and transmitting the medical text to a field within the second application via the UI feature.

In some examples, the UI feature is a browser extension and the second application is a web browser.

In some examples, the UI feature is accessible and usable in both the medical dictation application and the second application.

In some examples, the UI feature is a keyboard interface for receiving user inputs to a computing device on which the medical dictation application and the second application are operating.

Additional embodiments and features are set forth in part in the description that follows, and will become apparent to those skilled in the art upon examination of the specification and may be learned by the practice of the disclosed subject matter. A further understanding of the nature and advantages of the present disclosure may be realized by reference to the remaining portions of the specification and the drawings, which form a part of this disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The description will be more fully understood with reference to the following figures in which components are not drawn to scale, which are presented as various examples of the present disclosure and should not be construed as a complete recitation of the scope of the disclosure, characterized in that:

FIG. 1 illustrates a medical dictation application and various systems in communication with the medical dictation application through a network, in accordance with various embodiments of the disclosure.

FIG. 2 illustrates an example architecture for a dictation keyboard extension.

FIG. 3 illustrates an example architecture for a dictation application including a dictation keyboard extension.

FIG. 4A illustrates an example user interface for accessing a dictation keyboard extension.

FIG. 4B illustrates an example user interface including a dictation keyboard extension.

FIG. 5A illustrates an example user interface for configuration of a dictation keyboard extension.

FIG. 5B illustrates an example user interface including a dictation keyboard extension.

FIG. 5C illustrates an example user interface including a dictation keyboard extension.

FIG. 5D illustrates an example user interface including input provided using a dictation keyboard extension.

FIG. 6A illustrates an example user interface for a web browser utilizing a dictation keyboard extension.

FIG. 6B illustrates an example user interface for a web browser utilizing a dictation keyboard extension.

FIG. 6C illustrates an example user interface for a web browser utilizing a dictation keyboard extension.

FIG. 6D illustrates an example user interface for a web browser utilizing a dictation keyboard extension.

FIG. 7 is a schematic diagram of an example computer system implementing various embodiments in the examples described herein.

FIG. 8 illustrates a method of utilizing a dictation keyboard extension.

### DETAILED DESCRIPTION

Various customized OS or user interface (UI) features may be useful for specialized and secured applications, such as applications utilized to access and edit medical records. As such applications may have higher security requirements (e.g., the applications may be utilized to access sensitive or restricted data), existing methods of utilizing such customized OS or UI features may be insufficient as they may allow outside applications to access such data. For example, extensions utilizing the camera of a mobile device may allow photo applications to access sensitive data. Similarly, keyboard extensions may transmit data via the keyboard to other applications or utilities of a mobile device.

In some examples, a voice command integrated keyboard (e.g., a dictation keyboard extension) described herein may be used to access and edit medical records, such as for performing voice dictation. The voice command keyboard may be integrated into a larger application, such as voice dictation software, an electronic medical record (EMR), or the like. In some examples, a keyboard or other customized OS or UI feature may be integrated as a component of the larger application. In some examples, the keyboard or other customized OS or UI feature may be integrated into a larger application by sharing security features, such as a sandbox, sharing compute resources (e.g., a virtual machine), and/or configuring permissions between applications. Because the customized OS feature is integrated, the need for other integrations to access the customized OS feature, such as through various application programming interfaces (APIs) is eliminated, including for applications which execute in web browsers, such as mobile applications, and the like. The integration of a keyboard may, for example, allow providers to create dictation shortcuts available through the keyboard. Dictation shortcuts may include, for example, the ability to insert a large portion of text based on short commands available through the keyboard.

A customized OS feature or UI feature may, in various examples, be an extension to a mobile or other computing device features and may be sharable between various applications, web pages, and the like. For example, extensions to cameras or other utilities, For example, dictation keyboards, or other customized OS features may be additions and/or alternative to a OS features utilized by a mobile device. Such features may also be, in some examples, accessed via a web browser extension. For example, a keyboard may be utilized with medical dictation software. Such a keyboard may provide various features, such as the ability to insert large portions of text, to recognize and/or suggest common medical terminology. An OS or UI feature as used herein may be a feature providing user input to a device, such as tactile or audio input. Such OS or UI features are generally available from multiple applications executing at a computing device.

In various examples, customized OS or UI features, such as a keyboard may be persistent. That is, instead of timing out, the keyboard may require a command to exit the keyboard while utilizing the keyboard for dictation. Accordingly, the keyboard may be utilized for inserting large portions of text into, for example, dictated medical records. Further, because the customized OS features may be integrated into a larger application (e.g., a dictation application, an EMR, or the like), the keyboard may utilize authentication and security features of such applications, such as user credentials, multi factor authentication, encrypted communications, private networks, or the like. Accordingly, no data is transmitted between the application and another keyboard add on through API calls or other methods at the data layer of an application, such as a foreign language or symbolic keyboard. Such data transmission may jeopardize data security, not be compliant with medical privacy regulations, or may be associated with other risks.

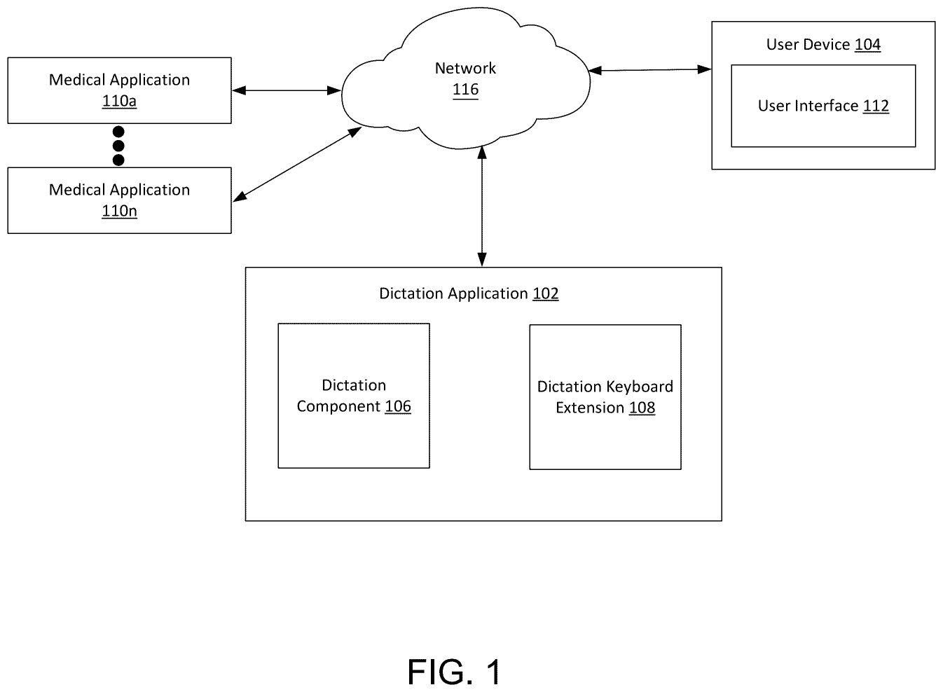

Various embodiments of the present disclosure will be explained below in detail with reference to the accompanying drawings. Other embodiments may be utilized, and structural, logical and electrical changes may be made without departing from the scope of the present disclosure. While the examples below are described with respect to a customized dictation keyboard, it is understood that the methods and systems described below may be applicable to other customized OS features. Turning now to the drawings, FIG. 1 illustrates an example dictation application 102 and various systems in communication with the dictation application 102 through a network 116, in accordance with various embodiments of the disclosure. Generally the dictation application 102 may be available to a user device 104 via a network 110. The user device 104 may further access other medical applications 110a-n via the network 110. The dictation application 102 includes at least a dictation component 106 and a dictation keyboard extension 108. In various examples, the dictation component 106 and the dictation keyboard extension 108 share data residing within the dictation application 102.

The dictation application 102 may generally be implemented by a computing device or combinations of computing resources in various embodiments. In various examples, the dictation application 102 may be implemented by one or more servers, cloud computing resources, and/or other computing devices. In some examples, the dictation application 102 may be implemented by one or more servers and computing resources at a user device (e.g., the user device 104). The dictation application 102 generally includes memory and/or storage locations to store program instructions for execution by the processor and various data utilized by the dictation application 102. The dictation application 102 may generally be an application with integrated dictation capabilities. For example, the dictation application 102 may be an application for generating medical records (e.g., operative notes, visit summaries, and the like), a medical records management system, an electronic health record (EHR), or the like. For example, the dictation application 102 may be an application for generation and transmission of operative notes, such as the application described is U.S. patent application Ser. No. 17/935,742, filed Sep. 27, 2022, entitled “Generation and Transmission of Operative Notes,” the disclosure of which is hereby incorporated by reference herein. In another example, the dictation application 102 may be an application providing coordinated access to medical records across multiple EHR systems, such at the system described in U.S. patent application Ser. No. 15/681,174, filed Aug. 18, 2017, entitled “Coordinated Mobile Access to Electronic Medical Records,” the disclosure of which is hereby incorporated by reference herein. The dictation application 102 generally includes a dictation component 106 and a dictation keyboard extension 108. The dictation keyboard extension may be utilized by the dictation component 106 and by other applications outside of the dictation application 102.

Medical applications 110a-n may similarly be implemented by one or more computing devices or combinations of computing resources in various embodiments. The medical applications 110a-n may be implemented by one or more servers, cloud computing resources, and/or other computing devices. In some examples, medical applications 110a-n may be implemented by one or more servers and computing resources at a user device (e.g., the user device 104). Medical applications 110a-n may be various types of applications including, for example, medical records management system, electronic health records (EHR), or the like.

The network 116 may be implemented using one or more of various systems and protocols for communications between computing devices. In various embodiments, the network 116 or various portions of the network 116 may be implemented using the Internet, a local area network (LAN), a wide area network (WAN), and/or other networks. In addition to traditional data networking protocols, in some embodiments, data may be communicated according to protocols and/or standards including near field communication (NFC), Bluetooth, cellular connections, and the like.

In various implementations, the user device 104 and/or additional user devices may be implemented using any number of computing devices including, but not limited to, a computer, a laptop, tablet, mobile phone, smart phone, wearable device (e.g., AR/VR headset, smart watch, smart glasses, or the like), smart speaker, vehicle (e.g., automobile), or appliance. A user accessing the dictation application 102 via a user device 104 may be a person utilizing a dictation application, such as a medical provider or other person associated with health care services. Generally, the user device 104 and/or other user devices accessing the dictation application 102 include one or more processors, such as a central processing unit (CPU) and/or graphics processing unit (GPU). The user device 104 may generally perform operations by executing executable instructions (e.g., software) using the processor(s).

Components of the dictation application 102 and in communication with the dictation application 102 shown in FIG. 1 are exemplary and may vary in some embodiments. For example, in some embodiments, the dictation application 102 may be distributed across multiple computing elements, such that components of the dictation application 102 communicate with one another through the network 116. In some embodiments, components of the dictation application 102 and/or medical applications 110a-n in communication with the dictation application 102 may execute at a user device 104. In some embodiments, components of the dictation application 102 and/or medical applications 110a-n may execute at a user device (e.g., user device 104). For example, a mobile client of the dictation application 102 and/or medical applications 110a-n may execute at the user device 104.

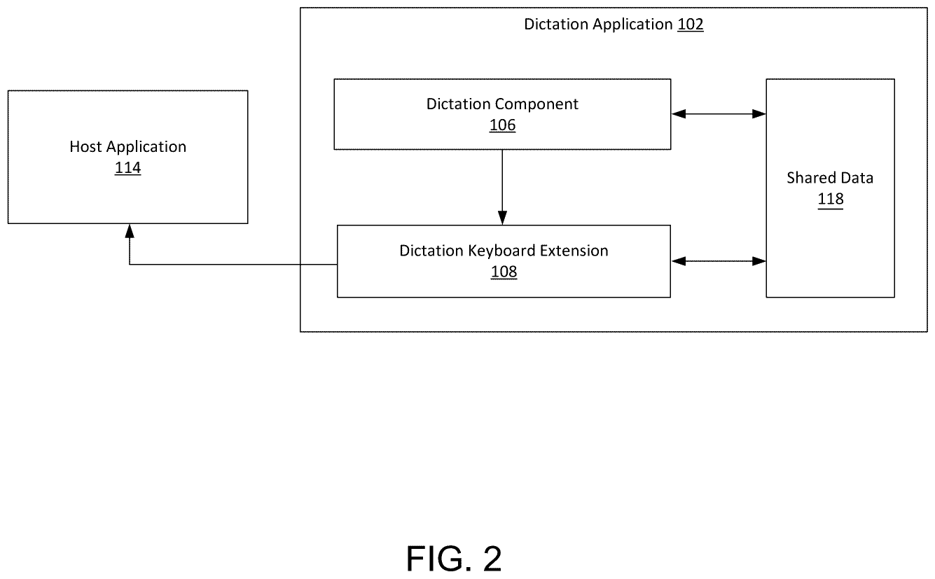

FIG. 2 illustrates an example architecture for a mobile device implementation of a user interface feature, which in one example is a dictation keyboard extension 108. The dictation keyboard extension 108 and the dictation component 106 are generally within a dictation application 102. A host application 114 is in communication with the dictation keyboard extension 108 such that the host application 114 may utilize the dictation keyboard extension 108. In various examples, the host application 114 and the dictation application 102 may execute, wholly or partially, at a user device, such as the user device 104. In various examples, the dictation application 102 may be in communication with and may be utilized by multiple host applications executing at a user device.

The dictation component 106 is generally a software application which utilizes dictation for text input. In various examples, the dictation component 106 may be a medical dictation application, such as an application utilized by medical providers to dictate medical notes, such as operative notes, notes from patient encounters, and the like. The dictation component 106 may utilize authentication methods such as two factor authentication, biometric input, and the like. In some examples, authentication methods utilized by the dictation component 106 may comply with various regulations to protect medical data.

In some examples, the dictation component 106 and the keyboard extension 108 may utilize shared authentication processes or methods of the dictation application 102. For example, because the dictation component 106 and the keyboard extension 108 are components of the dictation application 102, the dictation component 106 and the keyboard extension 108 may execute within a shared sandbox, execution environment (e.g., virtual machine), or the like. Accordingly, by authenticating with the dictation application 102, a user may be authenticated to access the dictation component 106 and the keyboard extension 108. For example, a user may provide credentials to access the dictation application 102 and, when authenticated, be provided with access to the dictation component 106 and the keyboard extension 108.

In some examples, authentication with the dictation application 102 may be standalone. That is, the user may authenticate with the dictation application 102 separately from authentication with other applications executing at the user device 104. In such examples, the user may be prompted to authenticate with the dictation application 102 when the dictation application is launched, such as when access to the dictation keyboard extension 108 is requested by a user. Such authentication may be provided by entering credentials, utilizing tokens, providing biometric input, or the like.

In some examples, authentication with the dictation application 102 may be tied to other applications executing at the user device 104 including, in some examples, the host application 114. For example, an EMR may be integrated with the dictation application 102 such that when a user signs into or otherwise authenticates with the EMR, the user is automatically also authenticated with the dictation application 102 through, for example, API data transfer between the EMR and the dictation application 102. Authentication with the dictation application 102 may further be provided through single sign on (SSO) or other methods of authenticating with multiple applications or services.

In some examples, the user may be provided, via such authentication, with access to the keyboard extension 108 across other applications executing on the user device. For example, an interface to the keyboard extension 108 in a second application may be non-functional when the user is not authenticated with the dictation application 102. The user may request to access the keyboard extension 108 within the second application and, after being authenticated with the dictation application 102, may be provided with a functional interface to the keyboard extension 108. In other examples, the interface to the keyboard extension 108 may only appear in the second application after the user is authenticated with the dictation application 102.

The dictation keyboard extension 108 generally allows dictation into text fields of various applications at the user device 104. For example, the dictation keyboard may include a selectable element which, when selected by a user, places the keyboard of the user device 104 into a dictation mode. In the dictation mode, audio input to the user device 104 (e.g., a user speaking into the device) causes text to be inserted into a field displayed at the user device 104.

In some examples, when the selectable element is selected by the user within a host application 114, the dictation application 102 may be launched for the dictation. The user may dictate using the dictation application 102 and copy text from the dictation application 102 into the selected field of the host application 114. To facilitate such copying, the dictation application 102 may, when launched, save a position of the cursor or selected field within the host application 114 (e.g., the cursor position may be cached within the dictation application 102). When the user copies text from the dictation application 102 to insert into the host application 114, the saved cursor position is utilized when switching back to the host application 114 such that the text is inserted at the saved cursor position.

The dictation keyboard 108 may further include one or more dictation shortcuts for entering text into a dictation. Dictation shortcuts may populate predefined text responsive to a particular audio input. Such predefined text may include single words or phrases (e.g., spelling out postoperative responsive to the user saying “post-op” into the user device 104) and/or longer defined text, such as sentences or paragraphs. For example, a user may say “normal procedure” into the user device 104, and a dictation field may be populated with a default description of a procedure. In some examples, dictation shortcuts may be utilized to provide dictation templates into a dictation. For example, dictation templates may provide an outline for a dictation and may include editable fields.

In some examples, the dictation keyboard extension 108 may be persistent. That is, the dictation keyboard 108 may remain at the user interface 112 of the user device 104 until the user provides a command to exit the dictation keyboard extension 108. For example, a verbal command may be used to close out the keyboard. In some examples, the dictation keyboard extension 108 may include a selectable element to close out the keyboard. Because the dictation keyboard extension 108 is persistent, the dictation keyboard extension 108 may be utilized to insert large portions of text into a dictation at the user device 104 without the keyboard timing out. In some examples, the dictation keyboard extension 108 may remain at the user interface 112 of the user device 104 only while within an application utilizing the dictation keyboard extension 108, and may terminate when the application is closed or executing in the background of the user device.

In other examples, the dictation keyboard extension 108 may persist across applications. That is, the dictation keyboard extension 108 may be visible and accessible even when exiting an application utilizing the dictation keyboard extension 108. Further, the dictation keyboard extension 108 may continue executing on the user device 104 when the dictation application 102 and/or the host application 114 are closed by a user. Such persistence may allow the user to remain authenticated at the host application 102 for some predefined period of time (e.g., 20 minutes) such that the dictation keyboard extension 108 may be accessed from other applications at the user device 104 without additional authentication of the user device 104. In such examples, when the dictation keyboard extension 108 is accessed from another application on the user device 104, the host application 102 may be launched when the dictation keyboard extension 108 is selected without additional authentication of the user device at the host application 102.

The dictation component 106 and the dictation keyboard extension 108 are components within the dictation application 102. Because the dictation keyboard extension 108 and the dictation component 106 are within the same application 102, the dictation keyboard extension 108 and the dictation component 106 can directly communicate with one another, without utilizing application programming interfaces (APIs) or other methods of communication between separate applications. Such direct communication may prevent data from being transmitted between the keyboard and an outside application, such that no data is transmitted between an outside application and another keyboard add on. Accordingly, the direct communication may protect sensitive medical and patient data, increasing data security.

Direct communication between the dictation component 106 and the keyboard extension 108 may include communication of data within a shared execution environment or sandbox of the dictation application 102. Because the data generally remains within the execution environment of the dictation application 102, the data is not exposed to other applications or processes of the user device when being communicated between the dictation component 106 and the keyboard extension 108. In some examples, additional or other methods may be utilized to protect data communicated between the dictation component 106 and the keyboard extension 108 such as advanced permissions, encryption, and the like.

Further, the dictation component 106 and the dictation keyboard extension 108 utilize shared data 118 within the dictation application 102. Shared data 118 may include, in various examples, dictation information such as dictation templates, dictation shortcuts, or other user settings related to dictation. In some examples, shared data 118 may further include patient data and/or instructions for obtaining patient data from another application or source. Patient data may include, in various examples, patient biographical data (e.g., name, identifier, or the like), diagnoses, medications, procedures, medical history, or the like. In some examples, such as where the dictation component 106 is an EMR, such patient information may be directly stored as shared data 118. In some examples, the dictation component 106 may be in communication with other medical applications (e.g., medical applications 110a-n) and such data may be obtained from another medical application. In such examples, the shared data 118 may include API calls or other data utilized to obtain such patient data from medical applications.

The host application 114 may be any application executing on the user device 104 including, for example, a web browser, other medical dictation application, e-mail application, or the like. In one example, the host application 114 may be a web browser utilized to access other services or applications utilizing dictation. The host application 114 may also directly access the dictation keyboard extension 108. For example, the host application 114 and the dictation application 102 may be configured such that when a user is authenticated with the dictation application 102, advanced permissions are configured between the host application 114 and the dictation application 102, such that the host application 114 may directly access the keyboard extension 108 without API calls or other methods which could expose data to applications or processes outside of the host application 114 and the dictation application 102. The host application 114 and the dictation application 102 may further communicate via a presentation layer of the respective applications, such that data is not transferred between the applications at the data layer. In some examples, before accessing the dictation keyboard extension 108, the user device 104 may be authenticated at the dictation application 102. In various examples, the dictation keyboard extension 108 may be accessed within the host application 114 through a keyboard menu, browser extension, or the like.

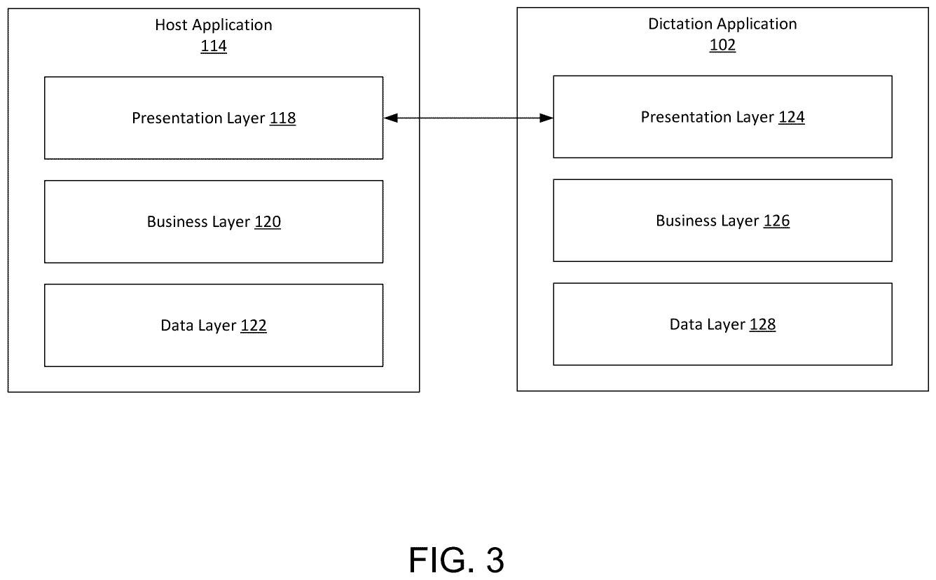

For example, FIG. 3 illustrates an example architecture for the dictation application 102 and the host application 114. The dictation application 102 generally includes a presentation layer 124, a business layer 126, and a data layer 128. The host application 114 similarly includes a presentation layer 118, a business layer 120, and a data layer 122. The dictation application 102 and the host application 114 generally communicate between the presentation layer 124 of the dictation application 102 and the presentation layer 118 of the host application 114. Accordingly, data is not transmitted between the data layer 128 and the data layer 122. Such communications at the presentation layer instead of the data layer may allow for easier integration between the dictation application 102 and the host application 114. For example, communication between the data layer 128 and the data layer 122 may require configuration of API calls that are particular to the dictation application 102 and/or the host application 114, meaning that configuring the dictation application 102 to integrate with other applications on the user device 104 could be a more cumbersome process than configuring communications at the presentation layer. Accordingly, communicating at the presentation layer may allow for more flexibility to use the dictation application 102 and the dictation keyboard extension 108 by other applications on the user device 104.

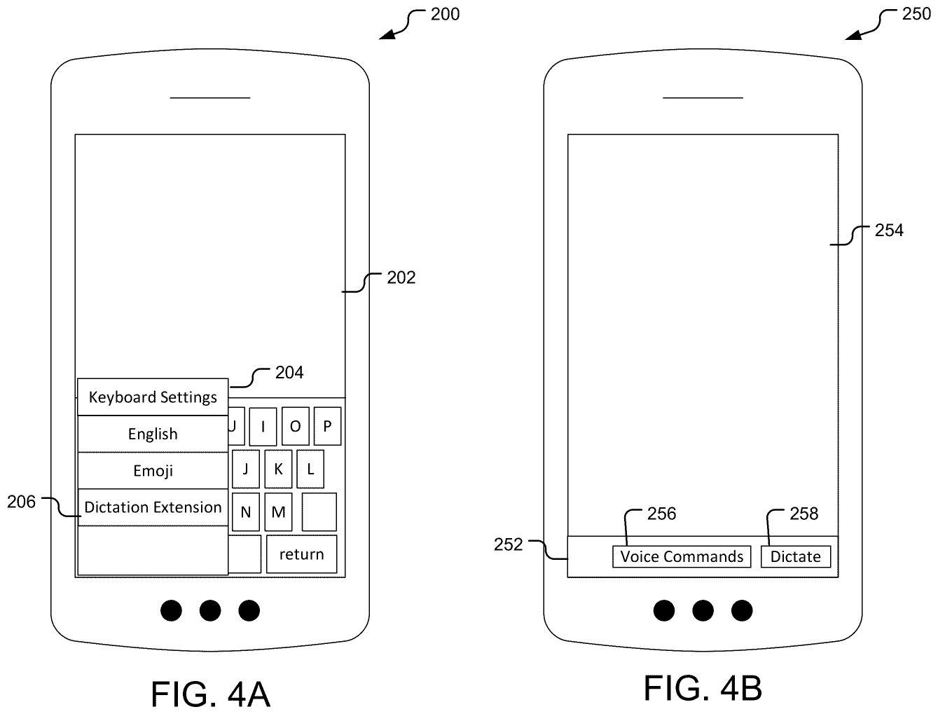

FIG. 4A illustrates an example user interface 200 for accessing a dictation keyboard extension. The user interface 200 may be an interface of the dictation component 106 of the dictation application 102 or within a host application 114 directly accessing the dictation keyboard extension 108 of the dictation application 102. In various examples, the user interface 200 may be displayed at the user interface 112 of the user device 104. The user interface 200 may be displayed responsive to a user selecting an editable field 202 at the user interface 112 of the user device 104, and selecting a menu 204 of a keyboard displayed at the user device 104. The menu 204 of the user interface 200 generally includes a selectable element 206 which, when selected, displays the interface to the dictation keyboard extension 108. In some examples, selecting the selectable element 206 may cause the dictation application 102 to launch, and the interface to the dictation keyboard extension 108 may be presented within the dictation application 102.

FIG. 4B illustrates an example user interface 250 including an interface to the dictation keyboard extension 252. The user interface 250 may be an interface of the dictation component 106 of the dictation application 102 presented within the dictation application 102 or within a host application 114 directly accessing the dictation keyboard extension 108. The user interface 250 may be displayed at the user interface 112 of the user device 104. The user interface 250 generally includes an editable field 254. The interface to the dictation keyboard extension 252 includes a selectable element 256 which, when selected, causes the dictation keyboard extension 108 to receive audio voice commands via the user device 104. The interface to the dictation keyboard extension 252 further includes a selectable element 258 which, when selected causes the dictation keyboard extension 108 to receive audio input via the user device 104 and display text in the editable field 254 responsive to the audio input.

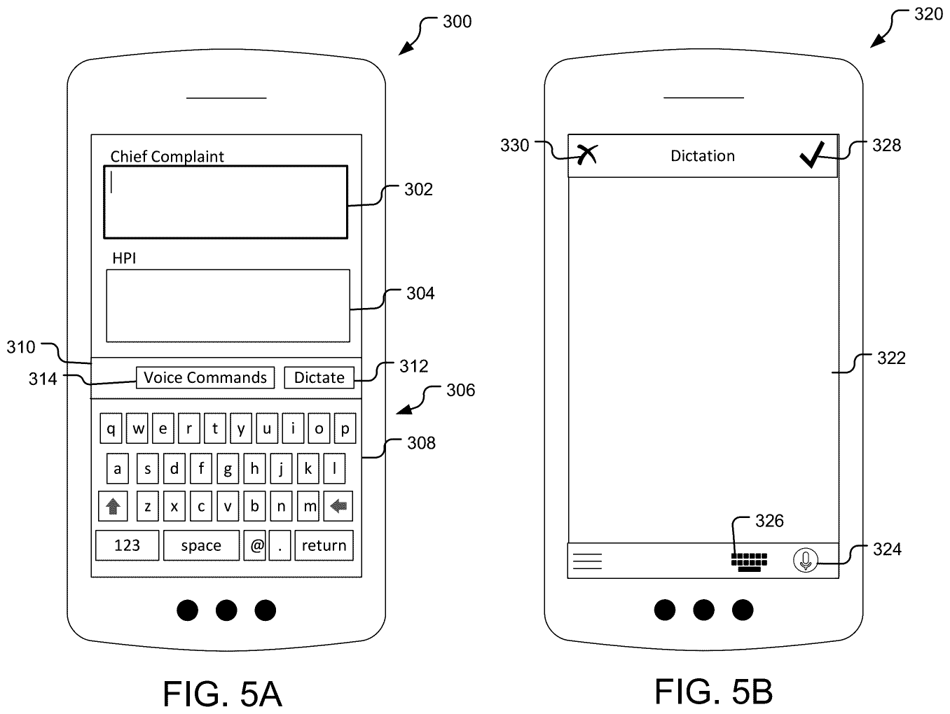

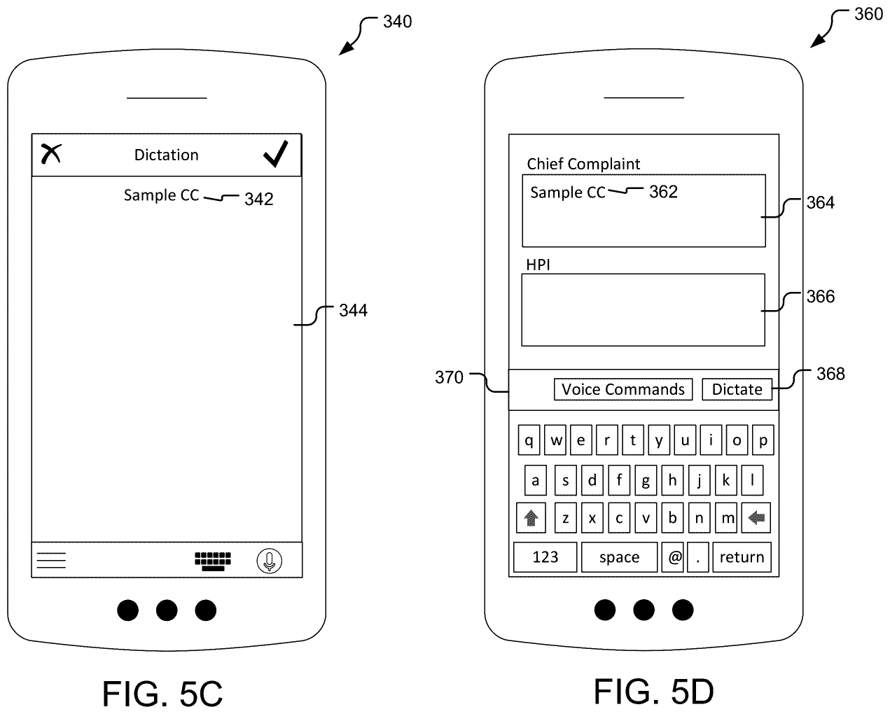

FIGS. 5A-5D illustrate example user interfaces presented at the user device 104 when utilizing the dictation keyboard extension 108. FIG. 5A illustrates an additional example user interface 300 for accessing a dictation keyboard extension 108. The user interface 300 may be an interface of the dictation component 106 of the dictation application 102 or within a host application 114 directly accessing the dictation keyboard extension 108. The user interface 300 may be displayed at the user interface 112 of the user device 104. The user interface 300 generally includes an editable field 302 and an editable field 304. In various examples, when one of the editable fields 302 and 304 is selected, a keyboard element 306 may be displayed at the user interface 300. The keyboard element 306 includes a keyboard 308 for providing manual (e.g., touch) input to the editable fields 302 and 304 and a interface to the dictation keyboard extension 310. The interface to the dictation keyboard extension 310 includes a selectable element 314 which, when selected, causes the dictation keyboard extension 108 to receive voice commands and a selectable element 312, which, when selected, causes the dictation keyboard extension 108 to receive audio input via the user device 104 to display text in a selected editable field 302.

In some examples, selecting the selectable element 314 or the selectable element 312 may launch the dictation application 102. In such examples, the user may, if not already authenticated at the dictation application 102, be directed to an interface for authenticating with the dictation application 102.

FIG. 5B illustrates an example user interface 320 including an interface to a dictation keyboard extension 108. In some examples, the user interface 320 may be presented at the user interface 112 of the user device 104 responsive to selectable component for providing dictation via a dictation keyboard extension 108. For example, the user interface 320 may be displayed responsive to selection of the selectable element 312 of the user interface 300. The user interface 320 may be a user interface of the dictation application 102 in various examples. That is, the user interface 320 may be presented to the user when the dictation application 102 is launched responsive to the user requesting to provide dictation using the dictation keyboard extension 108. The user interface includes an editable field 322. The editable field 322 may correspond to an editable field selected at the user interface 300 (e.g., editable field 322 may correspond to editable field 302). The user interface 320 includes a selectable element 324 to activate a microphone of or in communication with the user device 104. For example, when the selectable element 324 is selected, the user may dictate using the dictation keyboard extension 108. The user interface 320 further includes a selectable element 326 to access a keyboard for manual input. The user interface 320 further includes selectable elements 328 and 330 which may terminate or close out the dictation keyboard extension 108. For example, the selectable element 328 may, when selected, indicate that the dictation is complete while the selectable element 330 may close out the dictation keyboard extension 108 without completing the dictation. In some examples, selecting the selectable element 328 may cause the text within the editable field 322 to be copied for insertion into the editable field 302 or 304 of the user interface 300.

FIG. 5C illustrates an example user interface 340 including a dictation keyboard extension. The user interface 340 may generally be displayed at the user interface 112 of the user device 104 responsive to receiving audio input at the user interface 320. For example, text 342 is displayed in an editable field 344 responsive to receipt of audio input via the dictation keyboard extension 108.

FIG. 5D illustrates an example user interface 360 including input provided using a dictation keyboard extension 108. The user interface 360 is generally displayed at the user interface 112 of the user device 104. The user interface 360 may be presented responsive to a user selecting an element at a user interface (e.g., the user interface 340) indicating that the dictation is complete. For example, text 362 is displayed within an editable field 364 responsive to a user dictating into the editable field 364 using the dictation keyboard extension 108. The user interface 360 includes an additional editable field 366, where the user may dictate into the editable field 366 by, for example, selecting a selectable component 368 of an interface to the dictation keyboard extension 370.

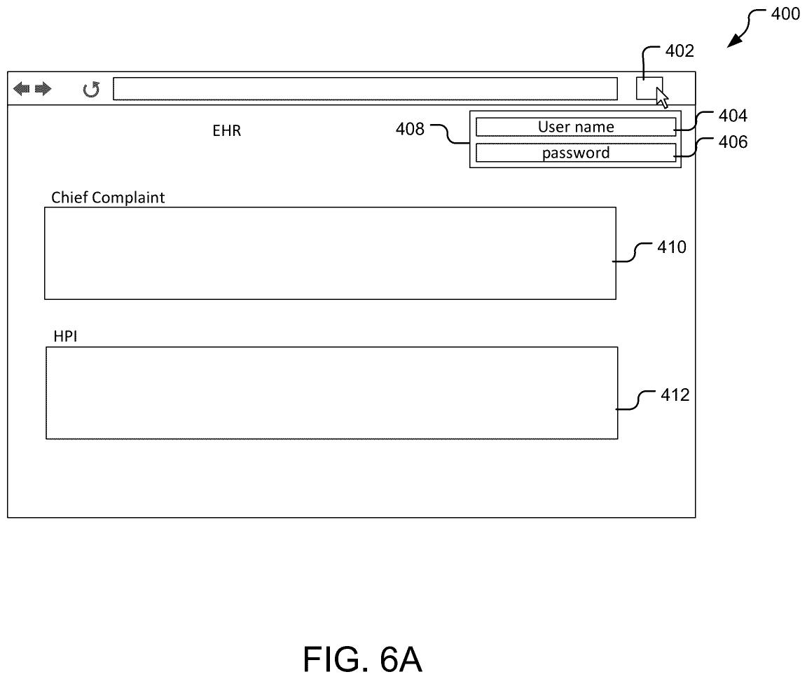

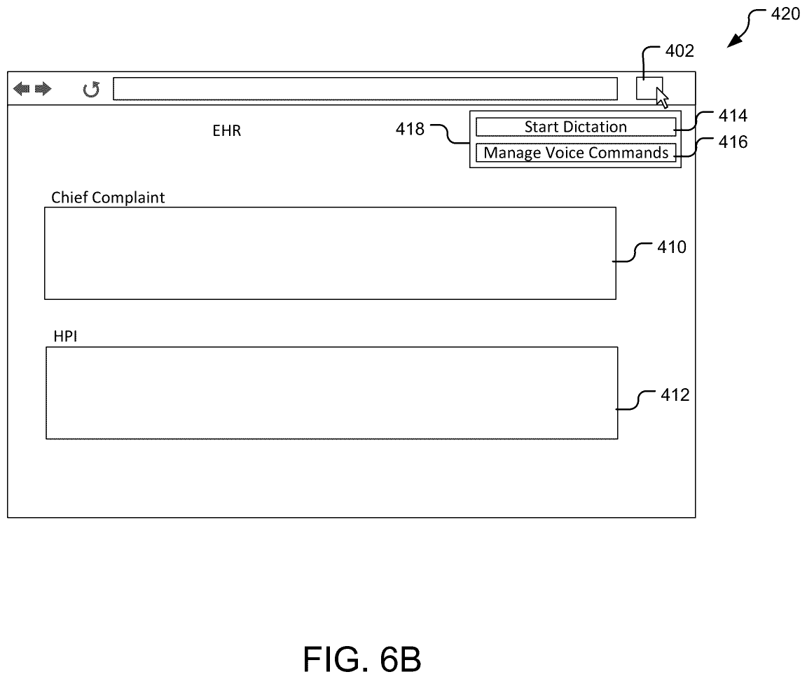

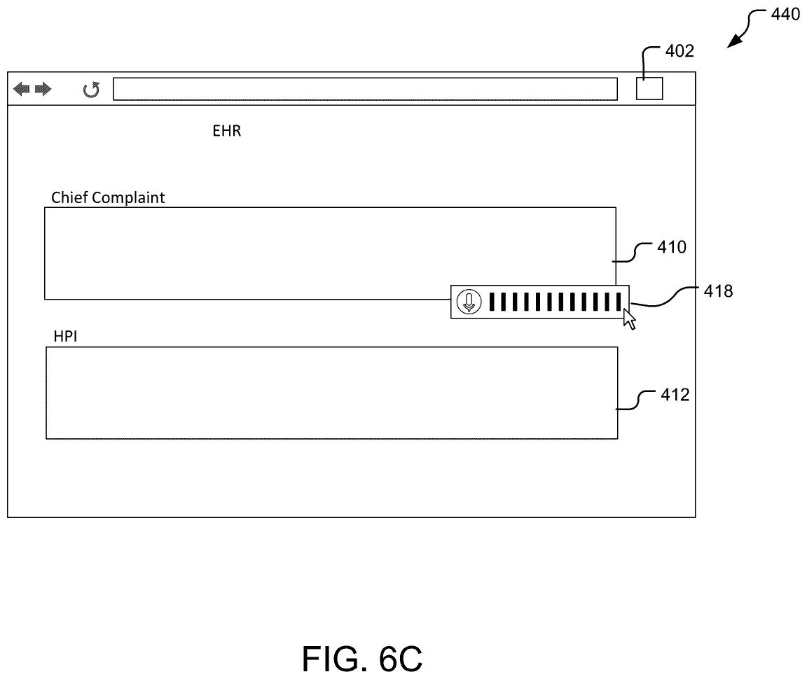

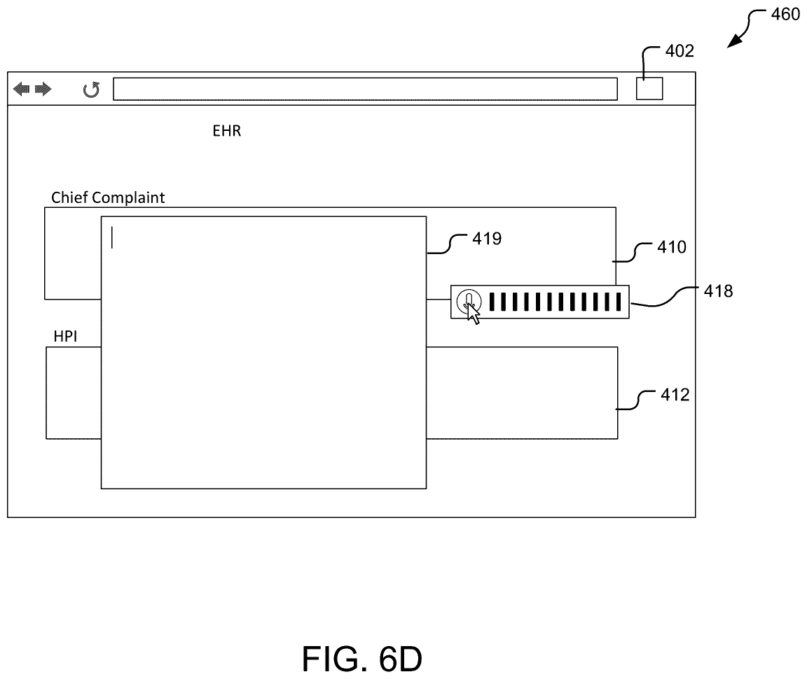

FIG. 6A illustrates an example user interface 400 for a web browser utilizing a dictation keyboard extension. The user interface 400 may be displayed, for example, at a user interface 112 of the user device 104. The user interface 400 may be an interface to a host application 114 (e.g., a secondary application) on the user device 104, where the host application 114 is a web browser. The web browser may be utilized to access external services or systems, such as EMRs, practice management systems, or the like. The user interface 400 includes an extension 402 which, when selected, causes display of an interface to the dictation keyboard extension 408. The interface to the dictation keyboard extension 408 includes fields 404 and 406 for entering credentials to access the dictation application 102. Such fields may be presented to the user when the user is not already authenticated at the dictation application 102. Though the fields 404 and 406 are shown as receiving a user name and password, other interfaces to the dictation keyboard extension may include elements for providing other types of authentication data, such as biometric input (e.g., facial recognition, fingerprints, and the like).

FIG. 6B illustrates an example user interface 420 for a web browser utilizing a dictation keyboard extension 108. The user interface 420 The interface to the dictation keyboard extension 408 includes a selectable element 414 which, when selected, may allow a user to dictate into an editable field 410 or 412 of the user interface 400. The interface to the dictation keyboard extension 408 further includes a selectable element 416 which, when selected, may allow a user to manage settings (e.g., voice commands) of the dictation keyboard extension 108.

FIG. 6C illustrates an example user interface 440 for a web browser utilizing a dictation keyboard extension 108. The user interface 440 may be presented within a web browser to a user after the selection of the selectable element 414 in the user interface 420. The user interface 440 includes a dictation control element 418 provided as an interface to the dictation keyboard extension 108. For example, as shown in FIG. 6D, selection of a microphone component of the dictation control element 118 may allow for a user to access the dictation keyboard extension 108 to dictate into the editable fields 410 or 412. In the example shown in the user interface 460 of FIG. 6D, such selection causes a window 419 to appear within the user interface 460. Dictated text may appear in the window 419 and, when the dictation is complete, may be copied into the editable field 410 or the editable field 412 of the user interface 460.

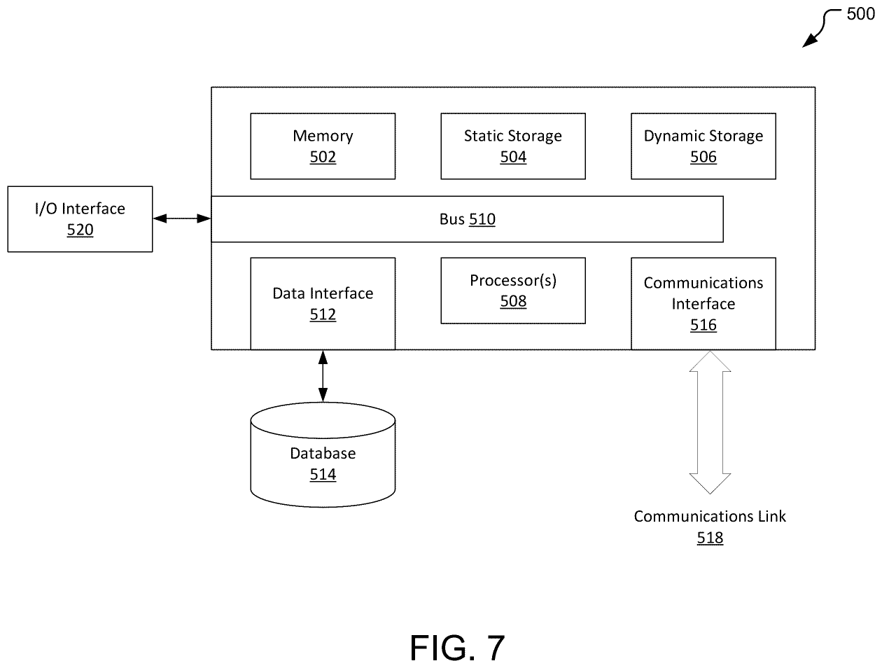

Turning to FIG. 7, an example computing system 500 may be used for implementing various embodiments in the examples described herein. For example, the dictation application 102 may be located at one or several computing systems 500. In various embodiments, user device 104 is also implemented by a computing system 500. Various medical applications 110a-n may also be implemented by one or several computing systems 500. This disclosure contemplates any suitable number of computing systems 500. For example, the computing system 500 may be a server, a desktop computing system, a mainframe, a mesh of computing systems, a laptop or notebook computing system, a tablet computing system, an embedded computer system, a system-on-chip, a single-board computing system, or a combination of two or more of these. Where appropriate, the computing system 500 may include one or more computing systems; be unitary or distributed; span multiple locations; span multiple locations; span multiple machines; span multiple data centers; or reside in a cloud, which may include one or more cloud components in one or more networks.

Computing system 500 includes a bus 510 (e.g., an address bus and a data bus) or other communication mechanism for communicating information, which interconnects subsystems and devices, such as processor 508, memory 502 (e.g., RAM), static storage 504 (e.g., ROM), dynamic storage 506 (e.g., magnetic or optical), communications interface 516 (e.g., modem, Ethernet card, a network interface controller (NIC) or network adapter for communicating with an Ethernet or other wire-based network, a wireless NIC (WNIC) or wireless adapter for communicating with a wireless network, such as a WI-FI network), input/output (I/O) interface 520 (e.g., keyboard, keypad, mouse, microphone). In particular embodiments, the computing system 500 may include one or more of any such components.

In particular embodiments, processor 508 includes hardware for executing instructions, such as those making up a computer program. The processor 508 circuity includes circuitry for performing various processing functions, such as executing specific software for perform specific calculations or tasks. In particular embodiments, I/O interface 520 includes hardware, software, or both, providing one or more interfaces for communication between computing system 500 and one or more I/O devices. Computing system 500 may include one or more of these I/O devices, where appropriate. One or more of these I/O devices may enable communication between a person and computing system 500.

In particular embodiments, communications interface 516 includes hardware, software, or both providing one or more interfaces for communication (such as, for example, packet-based communication) between computing system 500 and one or more other computer systems or one or more networks. One or more memory buses (which may each include an address bus and a data bus) may couple processor 508 to memory 502. Bus 510 may include one or more memory buses, as described below. In particular embodiments, one or more memory management units (MMUs) reside between processor 508 and memory 502 and facilitate accesses to memory 502 requested by processor 508. In particular embodiments, bus 510 includes hardware, software, or both coupling components of computing system 500 to each other.

According to particular embodiments, computing system 500 performs specific operations by processor 508 executing one or more sequences of one or more instructions contained in memory 502. Such instructions may be read into memory 502 from another computer readable/usable medium, such as static storage 504 or dynamic storage 506. In alternative embodiments, hard-wired circuitry may be used in place of or in combination with software instructions. Thus, particular embodiments are not limited to any specific combination of hardware circuitry and/or software. In various embodiments, the term “logic” means any combination of software or hardware that is used to implement all or part of particular embodiments disclosed herein.

The term “computer readable medium” or “computer usable medium” as used herein refers to any medium that participates in providing instructions to processor 508 for execution. Such a medium may take many forms, including but not limited to, nonvolatile media and volatile media. Non-volatile media includes, for example, optical or magnetic disks, such as static storage 504 or dynamic storage 506. Volatile media includes dynamic memory, such as memory 502.

Computing system 500 may transmit and receive messages, data, and instructions, including program, e.g., application code, through communications link 518 and communications interface 516. Received program code may be executed by processor 508 as it is received, and/or stored in static storage 504 or dynamic storage 506, or other storage for later execution. A database 514 may be used to store data accessible by the computing system 500 by way of data interface 512. In various examples, communications link 218 may communicate with, for example, user devices to display user interfaces to the dictation application 102.

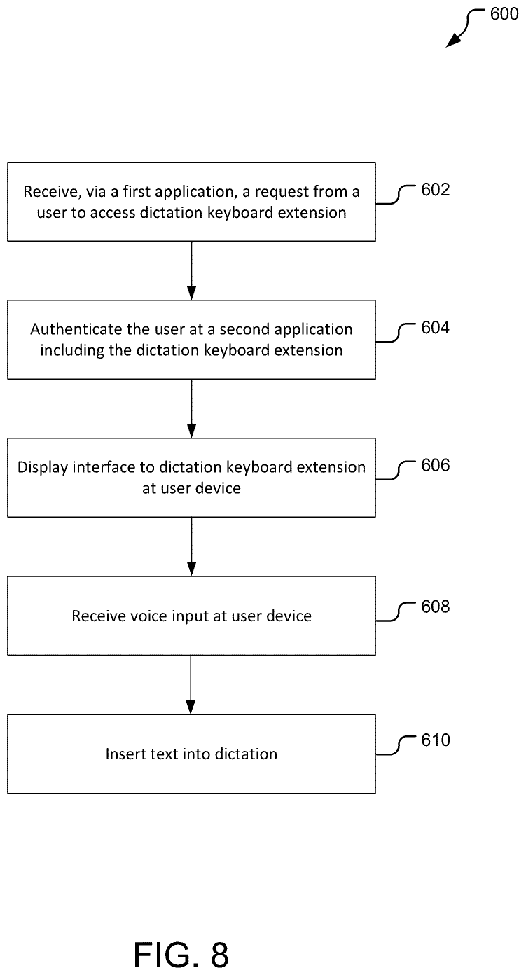

FIG. 8 illustrates an example method of utilizing a voice command integrated keyboard. At block 602, a request from a user device to access a dictation keyboard extension is received via a first application. The first application may be a host application 114 in communication with the dictation keyboard extension 108. The request may be received responsive to the user selecting an element of a keyboard menu (e.g., selectable element 206 of menu 204 at the user interface 200) to access an interface to the dictation keyboard extension. In another example, the request may be received responsive to a user selecting a web browser extension element (e.g., selectable element 402 at the user interface 400).

The user device is authenticated at a second application including the dictation keyboard extension 108 at block 604. For example, the second application may be a dictation application 102 requiring user authentication to access the dictation keyboard extension 108. Such authentication may be performed responsive to the user requesting access to the dictation keyboard extension 108. In some examples, the authentication may include determining whether the user device 104 is already signed into or authenticated at the dictation application 102. For example, the dictation keyboard extension 108 and/or the dictation application 102 may be persistent on the user device 104, such that the user device 104 remains authenticated with the dictation application 102 for some predetermined period of time even after exiting or closing the dictation application 102.

Where the user device 104 is not already signed into or authenticated at the dictation application 102, the user may be directed to a log in screen or may be otherwise authenticated at the dictation application 102. For example, the user may provide credentials to the dictation application 102, provide biometric input (e.g., facial recognition) to the dictation application 102, or utilize other authentication methods such as single sign on, secure tokens, and the like. Accordingly, in such examples, the dictation keyboard extension 108 and shared data 118 used by the dictation keyboard extension 108 may not be accessed by users who are not authorized to access the dictation application 102, increasing security of such data.

At block 606, an interface to the dictation keyboard extension is presented at the user device. For example, the interface to the dictation keyboard extension 252 is shown in the user interface 250. In various examples, the interface to the dictation keyboard extension 108 is provided within the dictation application 102 when the dictation application 102 is launched responsive to a request to access the dictation keyboard extension 108. When the dictation application 102 is launched, the dictation application 102 may save a position of a cursor or other marker within the host application 114.

Voice input is received at the user device at block 608. In some examples, the voice input may be received by the dictation keyboard extension 108 when a user selects a selectable element (e.g., selectable element 258 of the user interface 250) to begin dictation. Voice input may, in various example, include voice commands. Such voice commands may include dictation shortcuts to insert larger portions of text into the dictation responsive to a shorter spoken phrase. Such larger portions of text may include templates (e.g., templates for specific procedures), predefined text, and the like. Voice commands may further, in some examples, include requests to obtain patient information from shared data 118 and/or from external medical systems in communication with the dictation application 102. In some examples, the voice input may be the text to be inserted into the dictation.

At block 610, text is inserted into a dictation. For example, voice input or commands received at block 608 may be translated into text by the dictation component of the dictation application 102. In some examples, the dictation keyboard extension 108 may insert text into the dictation by accessing shared data 118 and/or external medical system in communication with the dictation application 102. For example, where the voice input is a voice command to insert a dictation template into the dictation, the dictation keyboard extension 108 may access shared data 118 to retrieve the dictation template from the shared data 118. In another example, the voice input may be a voice command to insert a patient name into a dictation. The dictation keyboard extension 108 may then access shared data 118 to query an external medical system (e.g., an EMR) to obtain a patient name to insert into the dictation.

In some examples, text may first be inserted into an interface at the dictation application 102 (e.g., user interface 340 shown in FIG. 5C). When the dictation is complete, the user may select a selectable element to copy the text of the dictation into an editable field within the host application 114. The text is then communicated from the dictation application 102 to the host application 114 via a copy and paste operation, such that the text is communicated between presentation layers of the dictation application 102 and the host application 114. The dictation application 102 may utilize a saved cursor position or location from the host application 114 to insert text into an editable field of the host application 114. When accessing the host application 114, the dictation application 102 may provide the saved cursor position to the host application 114 such that the dictated text is inserted into a last position or location of the cursor within the host application 114.

In accordance with the disclosure, the dictation keyboard extension may provide increased data security in medical dictation while providing dictation across applications of a user device. For example, the dictation keyboard extension may be a component of a larger dictation application, and may share an authentication process with the larger dictation application. The dictation keyboard extension may further be directly accessed by various applications on a user device, such that various interfaces which would require the transfer of data between a keyboard extension and an application are not required. Accordingly, less data is transferred between applications, increasing the security of such data.

The technology described herein may be implemented as logical operations and/or modules in one or more systems. The logical operations may be implemented as a sequence of processor-implemented steps directed by software programs executing in one or more computer systems and as interconnected machine or circuit modules within one or more computer systems, or as a combination of both. Likewise, the descriptions of various component modules may be provided in terms of operations executed or effected by the modules. The resulting implementation is a matter of choice, dependent on the performance requirements of the underlying system implementing the described technology. Accordingly, the logical operations making up the embodiments of the technology described herein are referred to variously as operations, steps, objects, or modules. Furthermore, it should be understood that logical operations may be performed in any order, unless explicitly claimed otherwise or a specific order is inherently necessitated by the claim language.

In some implementations, articles of manufacture are provided as computer program products that cause the instantiation of operations on a computer system to implement the procedural operations. One implementation of a computer program product provides a non-transitory computer program storage medium readable by a computer system and encoding a computer program. It should further be understood that the described technology may be employed in special purpose devices independent of a personal computer.

The above specification, examples and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A medical dictation application configured to operate on a computing device comprising:
a dictation component; and
a customized operating system (OS) feature in communication with the dictation component, wherein the customized OS feature and the dictation component utilize a shared authentication process of the medical dictation application, wherein the customized OS feature is inaccessible to a user device prior to authentication of the user device with the medical dictation application utilizing the shared authentication process.

2. The medical dictation application of claim 1, wherein the customized OS feature is a keyboard extension, wherein the keyboard extension provides at least one dictation shortcut for inserting text into a dictation responsive to a voice command provided using the keyboard extension.

3. The medical dictation application of claim 2, wherein an interface to the keyboard extension at the user device is persistent, such that the keyboard extension executes on the user device until a command is provided to exit the keyboard extension or until a predetermined time out period has elapsed.

4. The medical dictation application of claim 1, wherein the customized OS feature and the dictation component utilize shared data within the medical dictation application, wherein the shared data includes patient data.

5. The medical dictation application of claim 1, wherein customized OS feature and the dictation component utilize shared data within the medical dictation application, wherein the shared data includes instructions to access patient data from an external medical system.

6. The medical dictation application of claim 1, wherein a second application separate from the medical dictation application is in communication with the customized OS feature.

7. A computer implemented method comprising:
receiving, from a user device within a first software application, a request to access a keyboard extension of a medical dictation application, wherein the keyboard extension is accessible within a user interface of the first software application and in communication with a dictation component of the medical dictation application;
receiving, at an interface to the keyboard extension, a voice command, wherein the voice command includes a dictation shortcut for inserting text into a dictation within the first software application; and
inserting the text into the dictation within the first software application.

8. The computer implemented method of claim 7, wherein the text is inserted into the dictation by copying text from the interface to the keyboard extension within the medical dictation application to the first software application using a cursor position determined with the request to access the keyboard extension of the medical dictation application.

9. The computer implemented method of claim 7, wherein the interface to the keyboard extension is displayed within the medical dictation application responsive to authenticating the user device at the medical dictation application.

10. The computer implemented method of claim 7, wherein the keyboard extension and the dictation component utilize shared data, wherein the method further comprises accessing, by the keyboard extension responsive to the voice command, the shared data.

11. The computer implemented method of claim 7, wherein the keyboard extension and the dictation component utilize shared data, wherein the shared data includes instructions to access patient data from an external medical system.

12. The computer implemented method of claim 7, wherein the interface to the keyboard extension is persistent, such that the interface to the keyboard extension executes on the user device until a command is provided to exit the keyboard extension or until a predetermined time out period has elapsed.

13. The computer implemented method of claim 7, wherein the keyboard extension extends a keyboard of the user device, wherein the voice command, when received, is not shared with the keyboard of the user device or additional extensions to the keyboard of the user device.

14. A computer implemented method for medical dictation comprising:
authorizing a user device for access to a dictation application;
launching the dictation application based on a command received from a user interface (UI) feature via a second application different from the dictation application, wherein the UI feature is accessible in both the dictation application and the second application;
receiving voice commands at the dictation application;
translating the voice commands into text by the dictation component; and
transmitting the text to the second application via the UI feature.

15. The computer implemented method of claim 14, wherein the UI feature is further accessible in a third application different from the dictation application and the second application.

16. The computer implemented method of claim 14, further comprising:
saving a cursor location of user activity within the second application; and
transmitting the text to the saved cursor location.

17. A method for medical dictation comprising:
launching a medical dictation application by selecting a user interface (UI) feature accessible within a second application, wherein the second application is different from the medical dictation application;
receiving input by the medical dictation application via the UI feature;
translating the input into medical text by the medical dictation application;
providing the medical text to the UI feature; and
transmitting the medical text to a field within the second application via the UI feature.

18. The method of claim 17, wherein the UI feature is a browser extension and the second application is a web browser.

19. The method of claim 17, wherein the UI feature is accessible and usable in both the medical dictation application and the second application.

20. The method of claim 17, wherein the UI feature is a keyboard interface for receiving user inputs to a computing device on which the medical dictation application and the second application are operating.
